(19) United States
(12) Patent Application Publication
(10) Pub. No.: US 2024/0019419 A1
(43) Pub. Date: Jan. 18, 2024

(54) MICROFLUIDIC-BASED BLADDER CANCER MIMIC AND USE THEREOF

(71) Applicant: CHUNG ANG UNIVERSITY INDUSTRY ACADEMIC COOPERATION FOUNDATION, Seoul (KR)

(72) Inventors: In Ho CHANG, Seoul (KR); Joong Yull PARK, Seoul (KR); Se Young CHOI, Seoul (KR)

(21) Appl. No.: 18/036,909

(22) PCT Filed: Nov. 22, 2021
(86) PCT No.: PCT/KR2021/017186

§ 371 Date: May 15, 2023

(30) Foreign Application Priority Data
Feb. 4, 2021 (KR) 10-2021-0016235

(51) Int. Cl. G01N 33/50; C12N 5/071; C12M 3/00; B01L 3/00

(52) U.S. Cl. CPC G01N 33/5082; C12N 5/0697; C12M 21/08; B01L 3/502715; B01L 3/502707; G01N 33/5011; C12N 2513/00; B01L 2200/10; B01L 2400/082

(57) ABSTRACT

A microfluidic bladder cancer mimic and the use thereof is provided. The bladder cancer mimic is produced through 3D printing, which may more accurately reflect in vivo environments than conventional two-dimensional cell models and may be more simply produced than animal models. A method of screening bladder cancer treatment substances using a lab-on-a-chip including the bladder cancer mimic that may be used for the development of anticancer therapeutic agents and may also be used to develop patient-specific therapeutic agents by using patient-derived bladder cancer cells.

### TECHNICAL FIELD

The present invention relates to a microfluidic bladder cancer mimic and the use thereof.

### BACKGROUND

3D printers have been applied in various fields due to their broad-spectrum applicability, and in recent years, technology of forming a structure by printing cells has been studied. By using a 3D cell printer, it is possible to produce a more sophisticated structure compared to that obtained by conventional 3D cell culture technology using a pipette. A cell-friendly 3D cell structure may be formed using gelatin methacrylate which is harmless to cells. Thus, the cell structure may be used as a technology suitable for producing a brain-like chip by simulating a complex neural network structure.

Bladder cancer is one of the most common cancers, and there were an estimated 73,510 cases of bladder cancer in 2012. Bladder cancer is a disease that is more common in men than in women. Tumor formation occurs through a multistep process involving accumulation of genetic and epigenetic changes and networks. Genetic changes include changes in DNA sequence, which may be gain-of-function mutations (H-Ras and MYC oncogenes) or loss-of-function mutations (p53 and pRb tumor suppressor genes). Epigenetic changes include promoter hypermethylation of several tumor suppressor genes and the resulting suppression of expression (VHL and p16). In bladder cancer, many tumor suppressor genes such as BRCA1, WT1 and RARB are most frequently methylated. In bladder cancer, many additional important tumor suppressors and oncogenes may exist, and studies thereon are still in progress, and there has also been a steady demand for the development of screening technologies for bladder cancer therapeutic agents for these studies.

Meanwhile, as models for studying bladder cancer, planar cancer models have been used from the 1950s to the present. When comparing these models with in vivo responses, the planar cancer models lack the tumor microenvironment and it is difficult for such models to reflect the biological environment through co-culture. Thus, there has been a deficiency in the use of such planar cancer models. In addition, there have been attempts to produce bladder cancer models using mice in order to solve these problems, but it takes a long time to produce and establish the models, and differences between the animal models and humans still remain. Thus, there has been a need for a new bladder cancer model.

Accordingly, the present inventors have found a bladder cancer mimic and have found that the bladder cancer mimic may be used to screen agents for bladder cancer treatment or develop patient-specific agents for bladder cancer treatment, thereby completing the present invention.

### SUMMARY OF THE INVENTION

An object of the present invention is to provide a bladder cancer mimic comprising a stacked structure composed of an endothelial cell layer, a fibroblast layer and a bladder cancer cell layer.

Another object of the present invention is to provide a lab-on-a-chip comprising the bladder cancer mimic.

Still another object of the present invention is to provide a method for screening a substance for bladder cancer treatment, the method comprising steps of: (a) treating the lab-on-a-chip with a candidate substance for bladder cancer treatment; and (b) comparing a group treated with the candidate substance with a group treated with a control substance.

To achieve the above objects, the present invention provides a bladder cancer mimic comprising a stacked structure composed of an endothelial cell layer, a fibroblast layer, and a bladder cancer cell layer.

In one embodiment of the present invention, the endothelial cell layer may be composed of any one or more of bladder cancer patient-derived vascular endothelial cells and a human umbilical vein endothelial cell (HUVEC) line, the fibroblast layer may be composed of any one or more of bladder cancer patient-derived fibroblasts and MRC5, and the bladder cancer cell layer may be composed of any one or more of bladder cancer patient-derived bladder cancer cells, bladder cancer cell line T24, and bladder cancer cell line 5637.

In one embodiment of the present invention, the bladder cancer mimic may have a cylindrical shape.

In one embodiment of the present invention, the endothelial cell layer, the fibroblast layer, and the bladder cancer cell layer may be those obtained by 3D printing. In one embodiment of the present invention, the endothelial cell layer, the fibroblast layer, and the bladder cancer cell layer may each have a filling rate of 5 to 25%.

In one embodiment of the present invention, the permeate flow rate through the bladder cancer mimic may be 10 to 30 μl/min.

Another aspect of the present invention provides a lab-on-a-chip comprising the bladder cancer mimic.

Still another aspect of the present invention provides a method for screening a substance for bladder cancer treatment, the method comprising steps of: (a) treating the lab-on-a-chip with a candidate substance for bladder cancer treatment; and (b) comparing a group treated with the candidate substance with a group treated with a control substance.

Since the bladder cancer mimic produced by 3D printing and the lab-on-a-chip comprising the same according to the present invention each have a three-dimensional structure, they may more accurately reflect the in vivo environment compared to conventional two-dimensional cell models, and may also be produced more simply than animal models. In addition, the method of screening a substance for bladder cancer treatment using the same may be used for the development of anticancer therapeutic agents, and also be used to develop patient-specific therapeutic agents by using patient-derived bladder cancer cell lines.

### BRIEF DESCRIPTION OF DRAWINGS

FIG. 1 shows the configuration of each of a bladder cancer mimic and a lab-on-a-chip comprising the same according to the present invention. A bladder cancer mimic composed of bladder cancer cells, fibroblasts, and vascular cells is produced using a bio 3D printer, and is included in the lab-on-a-chip.

FIG. 2 shows a method for producing a portion of the bladder cancer mimic of the present invention. Specifically, a human umbilical vein endothelial cell (HUVEC) line as an endothelial cell layer, MRC5 as a fibroblast layer, and bladder cancer cell lines 5637 and T24 as a bladder cancer cell layer are separately cultured and mixed with the bio-ink GelMA, and each layer is printed in a cylindrical shape using a bio-printer, and then cured using UV light, thereby producing a bladder cancer mimic.

FIG. 3 shows a method of fabricating a lab-on-a-chip comprising the bladder cancer mimic of the present invention.

FIG. 4 shows the results of simulating the permeate flow rate depending on the shape of the bladder cancer mimic in Experimental Example 1.

FIG. 5 depicts photographs showing the results of analyzing cell viability depending on the filling rate of each cell layer in Experimental Example 2.

FIG. 6 depicts photographs showing the results of examining the permeate flow rate through the bladder cancer mimic in Experimental Example 3.

FIG. 7 graphically depicts the results obtained in Experimental Example 4 by co-culturing the cell layers of the bladder cancer mimic of the present invention in a Conditions for creating microfluids, measuring the cell proliferation rate of each cell layer, and comparing the measured cell proliferation rate with that of each of a group in which each cell layer was cultured alone and a group in which the cell layers were co-cultured without the Conditions for creating microfluids.

FIGS. 8 and 9 show the results obtained in Experimental Example 5 by counting the number of monocytic THP-1 cells migrated and analyzing the time-dependent expression level of cytokine (IFN-γ).

### DETAILED DESCRIPTION OF THE INVENTION

One aspect of the present invention provides a bladder cancer mimic comprising a stacked structure composed of an endothelial cell layer, a fibroblast layer, and a bladder cancer cell layer.

In one embodiment of the present invention, the endothelial cell layer may be composed of any one or more of bladder cancer patient-derived vascular endothelial cells and a human umbilical vein endothelial cell (HUVEC) line, the fibroblast layer may be composed of any one or more of bladder cancer patient-derived fibroblasts and a fibroblast line (MRC5), and the bladder cancer cell layer may be composed of any one or more of bladder cancer patient-derived bladder cancer cells, bladder cancer cell line T24, and bladder cancer cell line 5637.

Specifically, the endothelial cell layer may be composed of the human umbilical vein endothelial cell (HUVEC) line, the fibroblast layer may be composed of MRC5, and the bladder cancer cell layer may be any one of the bladder cancer cell line T24, the bladder cancer cell line 5637, and the patient-derived bladder cancer cells. Alternatively, the endothelial cell layer may be composed of the patient-derived vascular endothelial cells, the fibroblast layer may be composed of the bladder cancer patient-derived fibroblasts, and the bladder cancer cell layer may be composed of the patient-derived bladder cancer cells

In one embodiment of the present invention, the bladder cancer mimic may have a cylindrical shape. As the bladder cancer mimic of the present invention has a cylindrical shape rather than a square column shape, as described below, the permeate flow rate through the mimic may be 10 to 30 μl/min, specifically 15 to 25 μl/min, more specifically 20 μl/min, and thus the bladder cancer mimic may be suitable for cell culture (FIG. 4).

In one embodiment of the present invention, the endothelial cell layer, the fibroblast layer, and the bladder cancer cell layer may be those obtained by 3D printing. The term “3D printing” means scanning a target structure in three dimensions to obtain an image and making the scanned image into a three-dimensional structure through cells and bio-ink. Each of the cell layers may further comprise a known material necessary for constituting the bio-ink that is used for 3D printing, and specifically, it may further comprise argarose, alginate, chitosan, collagen, decellularized extracellular matrix, fibrin/fibrinogen, gelatin, graphene, hyaluronic acid, hydroxyapatite, polycaprolactone (PCL), polylactic acid (PLA), poly-D,L-lactic-co-glycolic acid (PLGA), gelatin methacryloyl (GelMA), and/or pluronic F 127, without being limited thereto.

In one embodiment of the present invention, the endothelial cell layer, the fibroblast layer, and the bladder cancer cell layer may each have a filling rate of 5 to 25%, specifically 10 to 20%, more specifically 15%. The term “filling rate” refers to the area percentage of bioprinting ink corresponding to one layer relative to the bottom area of one 3D cell structure made by stacking 10 layers, when 3D printing a mixture of each cell line mixed with GelMA at a predetermined ratio. As the endothelial cell layer, the fibroblast layer, and the bladder cancer cell layer of the present invention are produced to each have a filling rate within the above-described range, there is an effect of increasing the viability of the cells (FIG. 5).

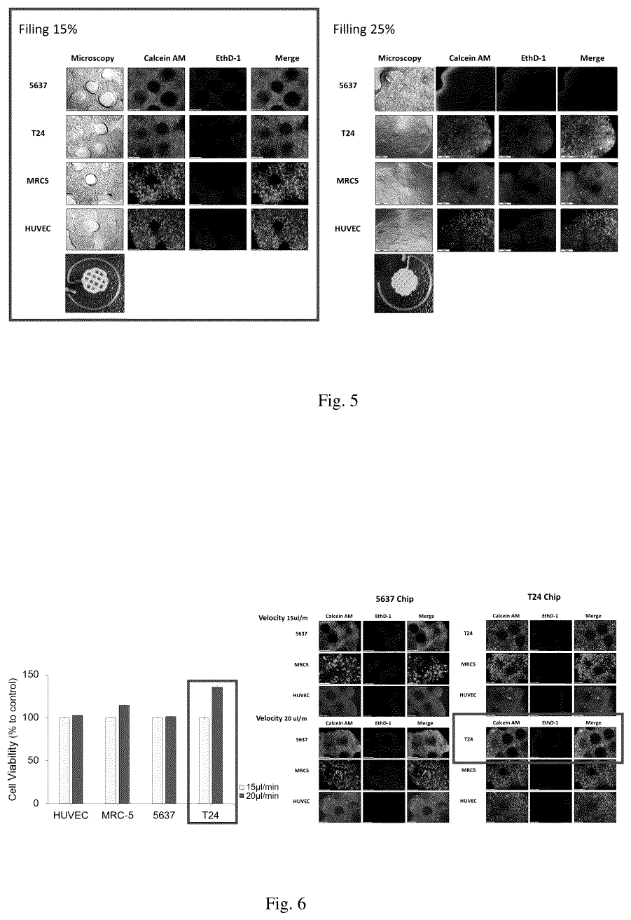

In one embodiment of the present invention, the permeate flow rate through the bladder cancer mimic may be 10 to 30 μl/min, specifically 15 to 25 μl/min, more specifically 20 μl/min. As the bladder cancer mimic has a cylindrical shape as described above, the permeate flow rate may be within the above-described range, and thus there is an effect of increasing the cell viability of the bladder cancer cell line in the bladder cancer mimic (FIG. 6).

Since the bladder cancer mimic produced by 3D printing according to the present invention has a three-dimensional structure, it may more accurately reflect the in vivo environment than conventional two-dimensional cell models, and may also be produced more simply than animal models. In addition, a lab-on-a-chip comprising the same and a method of screening a substance for bladder cancer treatment using the same may be used for the development of anticancer therapeutic agents, and also be used to develop patient-specific therapeutic agents by using patient-derived bladder cancer cell lines.

The present invention also provides a lab-on-a-chip comprising the bladder cancer mimic.

The lab-on-a-chip is a chip capable of analyzing reaction patterns of an analysis target substance with various biomolecules or sensors integrated on the chip while passing a small amount of the substance.

The structure and components of the lab-on-a-chip of the present invention may be those known in the art, except for the bladder cancer mimic, and may change depending on the intended use of the lab-on-a-chip. The lab-on-a-chip of the present invention may consist of, from bottom to top, a bottom casing, a bottom layer, a membrane, a middle layer on which the bladder cancer mimic may be placed, a top layer, and a top casing. Specifically, considering the purpose of detecting a candidate substance for bladder cancer treatment, the lab-on-a-chip of the present invention may be produced by placing the bottom layer on the bottom casing, and then placing a culture medium containing a mixture of THP-1 cells (2×10⁴ cells) and 20 ng/ml of PMA as a monocyte activator in the bottom layer, covering the upper side of the bottom layer with a membrane, and placing the middle layer and the bladder cancer mimic of the present invention thereon, followed by covering with the top layer and the top casing.

The present invention also provides a method for screening a substance for bladder cancer treatment, the method comprising steps of: (a) treating the lab-on-a-chip with a candidate substance for bladder cancer treatment; and (b) comparing a group treated with the candidate substance with a group treated with a control substance.

Step (a) is a step of treating the lab-on-a-chip with a candidate substance for bladder cancer treatment. Here, the lab-on-a-chip and the bladder cancer mimic included in the lab-on-a-chip may be the same as those described above, and the properties of the substance for bladder cancer treatment may be reflected depending on the components of the bladder cancer cell layer included in the bladder cancer mimic. Specifically, when the bladder cancer cell layer is any one of the bladder cancer cell line T24 and the bladder cancer cell line 5637, it may detect a non-specific bladder cancer treatment substance, and when the bladder cancer cell layer is composed of patient-derived bladder cancer cells, it may detect a patient-specific bladder cancer treatment substance.

Step (b) may be a step of comparing a group treated with the candidate substance with a group treated with a control substance. The control group may be a group in which the bladder cancer mimic is treated with a previously known bladder cancer treatment substance, or may be a group in which the bladder cancer mimic is not treated with the candidate substance for bladder cancer treatment. When the bladder cancer mimic is not treated with the candidate substance for bladder cancer treatment, it may be treated with a known substance within a range that does not inhibit or increase the physiological activity of the bladder cancer mimic.

In addition, the comparison of the group treated with the candidate substance with the group treated with the control substance may be performed by confirming the growth inhibition or death of the bladder cancer cell layer in the bladder cancer mimic or examining a bladder cancer growth inhibition or death marker released through the bladder cancer mimic.

In addition, the screening method may further comprise step (c) of selecting a bladder cancer treatment substance. In step (c), when the growth inhibition or death of the bladder cancer cell layer in the bladder cancer mimic or a bladder cancer growth inhibition or death marker released through the bladder cancer mimic is confirmed through the comparison performed in step (b) above, the candidate substance may be selected as a bladder cancer treatment substance. In addition, in the case of treatment with a conventional bladder cancer treatment substance as the control substance as described above, when the effect in the group treated with the candidate substance is improved compared to that in the group treated with the control substance, the candidate substance may be determined to have an improved effect compared to the conventional bladder cancer treatment substance.

### Embodiments of Invention

Hereinafter, one or more embodiments will be described in more detail with reference to examples. However, these examples are intended to illustrate one or more embodiments, and the scope of the present invention is not limited to these examples.

### Experimental Example 1: Permeate Flow Rate Simulation Depending on Shape of Bladder Cancer Mimic

The permeate flow rate depending on the shape of the bladder cancer mimic of the present invention was simulated to determine the optimal mimic shape.

Specifically, a computational fluid dynamics (CFD) technique was used to predict and visualize the flow inside the bladder cancer mimic. In the results, a higher flow rate indicates that medium supply is smoother, and a lower flow rate indicates that medium supply is not smooth.

As a result, as shown in FIG. 4, it could be confirmed that, when the bladder cancer mimic had a cylindrical shape, the permeation of medium occurred smoothly at 20 μl/min among permeate flow rates of 15 μl/min and 20 μl/min, and when the bladder cancer mimic had a square columnar shape, the area where the permeation of medium was not smooth at the same permeate flow rate of 20 μl/min was large. These results suggest that the cylindrical shape is the optimal shape of the bladder cancer mimic.

### Example 1: Production of Bladder Cancer Mimic

The HUVEC cell line was used as a cell line for a vascular endothelial cell layer, MRC5 was used as a cell line for a fibroblast layer, and bladder cancer cell lines 5647 and T24 were used as cell lines for a bladder cancer cell layer. Each of the cell lines was cultured, and GelMA was mixed with the culture medium containing each cell line, thus preparing bio-inks. Each bio-ink was 3D-printed into each cell layer by using a 3D printer, and each printed material was cured by UV irradiation. Next, the resulting vascular endothelial cell layer, fibroblast layer and bladder cancer cell layer were stacked together, thereby producing a bladder cancer mimic of the present invention.

### Experimental Example 2: Establishment of Filling Rate Condition

The viability of cells in the cell layers produced in Example 1 was analyzed depending on the conditions of the bio-ink and the filling rate of each cell layer.

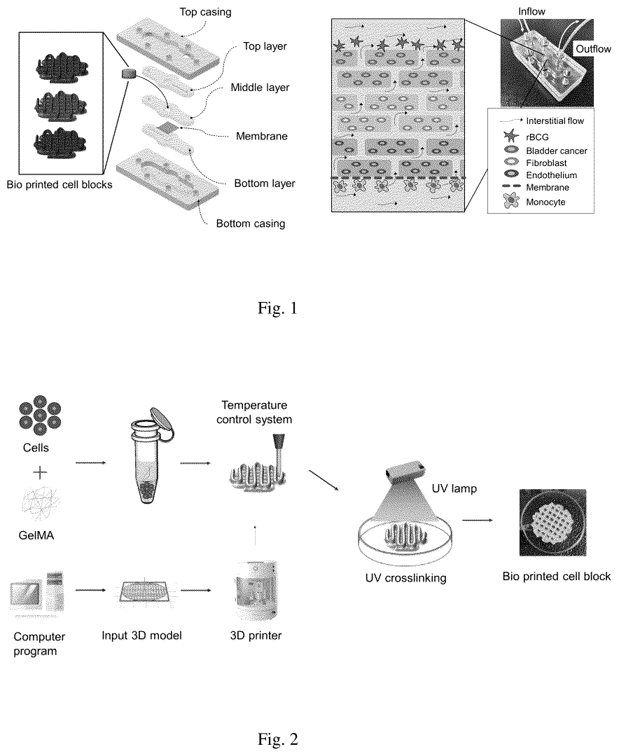

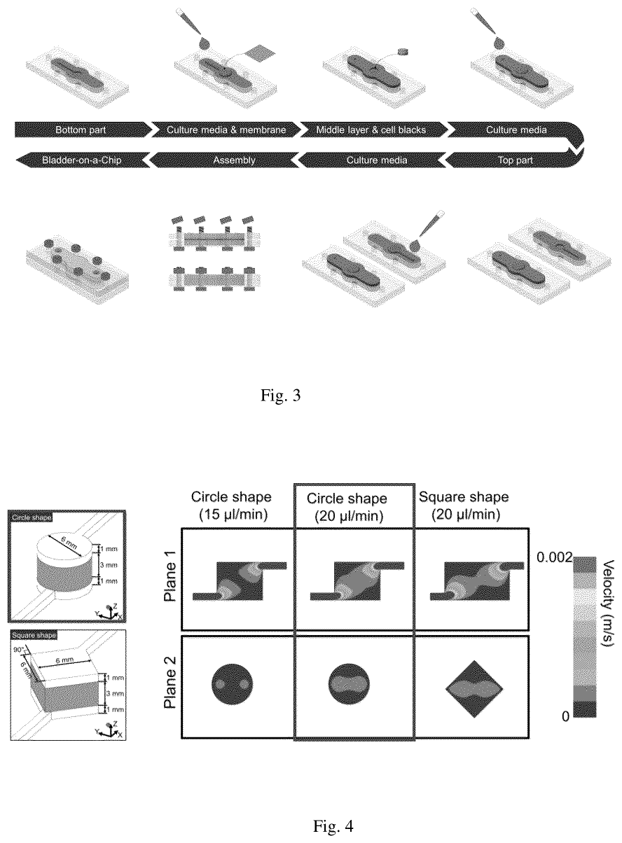

The vascular endothelial cell (HUVEC) line, the fibroblast cell line MIRC5, and the bladder cancer cell lines 5637 and T24, which were each mixed with GelMA, were each made into a 3D cell structure by printing with a printer set to have a filling rate of 15% or 25%, and a bladder cancer mimic was produced according to the method shown in FIG. 1. After 72 hours, the viability of cells in each 3D cell structure was analyzed by the cell counting kit-8 (CCK-8; Dojindo, MD, USA) method and the LIVE/DEAD staining method (Thermofisher, MA, USA), and the cell viabilities at the filling rates were compared.

As a result, as shown in FIG. 5, it was confirmed that the viability of cells in each cell layer was higher when the filling rate was 15% than when the filling rate was 25%.

### Experimental Example 3: Establishment of Permeate Flow Rate Condition for Bladder Cancer Mimic

Cell viability in the bladder cancer mimic depending on the flow rate of medium through the bladder cancer mimic was examined.

After a bladder cancer mimic was produced using the 3D cell structures produced to each have a filling rate of 15% established in Experimental Example 2, a culture medium was permeated through the bladder cancer mimic at a flow rate of 15 μl/min or 20 μl/min by means of a syringe pump. After 72 hours, the viability of cells in each 3D cell structure was analyzed by the cell counting kit-8 (CCK-8; Dojindo, MD, USA) method and the LIVE/DEAD staining method (Thermofisher, MA, USA), and the cell viabilities at the flow rates were compared.

As a result, as shown in FIG. 6, it was confirmed that the viability of cells in each cell layer was higher when the permeate flow rate was 20 μl/min than when the permeate flow rate was 15 μl/min.

### Experimental Example 4: Establishment of Conditions for Co-Culture of Cell Layers of Bladder Cancer Mimic

The cell viability when the cell layers of the bladder cancer mimic of the present invention were co-cultured in a Conditions for creating microfluids was compared with the cell viability shown when the cell layers were co-cultured without the microfluidic chip and the cell viability shown when each of the cell layers was cultured alone.

After 3D cell structures were produced, they were divided into a group in which each 3D cell structure was cultured alone (mono-culture group), a group in which the 3D cell structures were co-cultured (co-culture group), and a group in which the 3D cell structures were co-cultured in a microfluidic chip (co-culture plus microfluidic chip group). The mono-culture group and the co-culture group were each cultured in a 60-mm dish without the microfluidic chip. In the case of the co-culture plus microfluidic chip group, a culture medium was permeated at a flow rate of 20 μl/min through the bladder cancer mimic shown in FIG. 1. After 3 hours, 6 hours and 24 hours, each culture medium was collected, and the time-dependent concentration of each growth factor was measured by Luminex assay using the MAGPIX system and compared between the groups. The growth factors analyzed were TGF-beta 1, GM-CSF, PDGF-AA, and VEGF. To confirm cell viability, after 72 hours, the viability of cells in each 3D cell structure was analyzed by the cell counting kit-8 (CCK-8; Dojindo, MD, USA) method and the LIVE/DEAD staining method (Thermofisher, MA, USA) and compared between the groups.

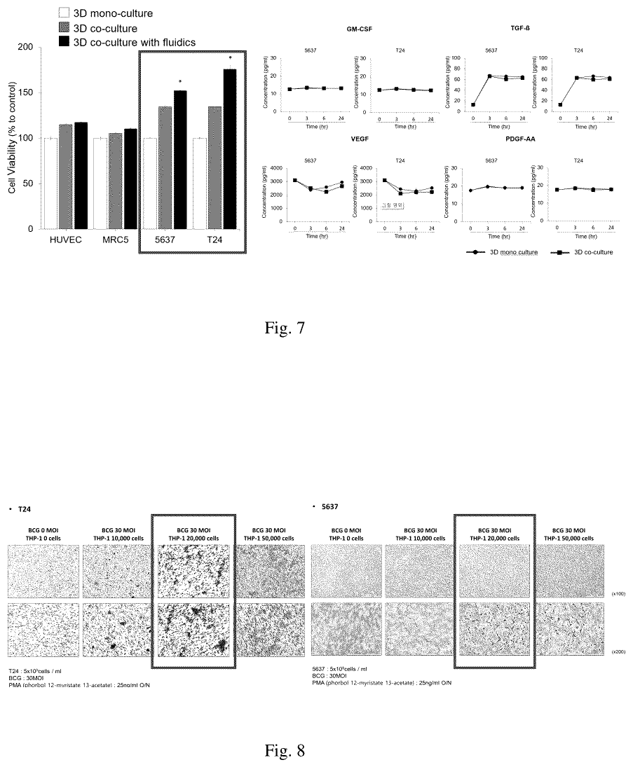

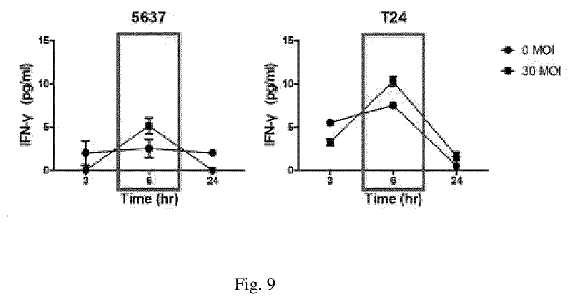

As a result, as shown in FIG. 7, it was confirmed that the cell viability in each cell layer was higher when the cell layers of the bladder cancer mimic of the present invention were co-cultured in the microfluidic chip than when the cell layers were co-cultured without the microfluidic chip and when each cell layer was cultured alone. In particular, it was confirmed that the bladder cancer cell lines 5637 and T24 had significantly high viability when they were co-cultured in the microfluidic chip.

### Experimental Example 5: Analysis of Level of Monocyte Permeation Through Bladder Cancer Mimic and Time-Dependent Expression of Cytokine

In order to confirm the biomimetic level of the bladder cancer mimic of the present invention, the present inventors examined the level of monocyte permeation through the bladder cancer mimic and whether or not an immune response such as time-dependent cytokine expression was induced.

In order to count the number of monocytic THP-1 cells migrated into the bladder cancer mimic, THP-1 cells were allowed to differentiate into macrophages for 24 hours in a culture medium supplemented with 25 nM of phorbol 12-myristate 13-acetate (PMA), and then various numbers (1×10⁴, 2×10⁴, and 5×10⁴) of THP-1 cells were injected into the bottom layer shown in FIG. 1. Thereafter, the upper side of the bottom layer was covered with a polycarbonate track etched (PCTE) membrane, and then the 3D cell structures were arranged in order on the middle layer, followed by treatment with 30 MOI of BCG, thereby producing a bladder cancer mimic. A culture medium was permeated through the bladder cancer mimic at a flow rate of 20 μl/min established in Experimental Example 3, and after 3 hours, 6 hours and 24 hours, the culture medium was collected and the time-dependent expression of cytokine was measured by Luminex assay using the MAGPIX system and compared. The PCTE membrane was separated 24 hours after the start of the experiment, fixed with 4% praformaldehyde, and then stained with 0.1% crystal violet, and the number of THP-1 cells migrated was counted under microscopic observation.

As a result, migration of the THP-1 cell line into the bladder cancer mimic was confirmed (FIG. 8), and an increase in the expression level of IFN-γ was confirmed by 6 hours of reaction. These results confirmed that the bladder cancer mimic of the present invention underwent an immune response, suggesting that the bladder cancer mimic of the present invention may be used as a bladder cancer model and may be used in a method for screening a bladder cancer treatment substance.

Final Conditions for Bladder Cancer Mimic

Taking the above results together, the method for producing the bladder cancer mimic and lab-on-a-chip of the present invention is summarized as follows:

The HUVEC line was used as a cell line for a vascular endothelial cell layer, MRC5 was used as a cell line for a fibroblast layer, and the bladder cancer cell line 5647 or T24 was used as a cell line for a bladder cancer cell layer. Each of the cell lines was cultured, and GelMA was mixed with the culture medium containing each cell line, thus preparing bio-inks. Each bio-ink was 3D-printed into each cell layer having a filling rate of 15% by using a 3D printer, a diameter of 6 mm and a height of 0.8 mm. Next, the resulting endothelial cell layer, fibroblast layer and bladder cancer cell layer were stacked together from bottom in a cylindrical shape, thereby producing a bladder cancer mimic.

After the bottom layer was placed on the bottom casing, a culture medium containing a mixture of THP-1 cells (2×10 4) and ng/ml of PMA as a monocyte activator was placed in the bottom layer whose upper side was then covered with a membrane. Next, the middle layer and the bladder cancer mimic of the present invention were placed thereon, followed by covering with the top layer, BCG treatment, and covering with the top casing, and the resulting structure was fixed with screws, thereby producing a lab-on-a-chip. Then, the culture medium was allowed to flow through the produced lab-on-a-chip at a permeate flow rate of 20 μl/min.

So far, the present invention has been described with reference to the preferred embodiments thereof. Those of ordinary skill in the art to which the present invention pertains will appreciate that the present invention may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

1. A bladder cancer mimic comprising a stacked structure including an endothelial cell layer, a fibroblast layer, and a bladder cancer cell layer.

2. The bladder cancer mimic of claim 1, wherein
the endothelial cell layer includes any one or more of bladder cancer patient-derived vascular endothelial cells and a human umbilical vein endothelial cell (HUVEC) line,
the fibroblast layer includes any one or more of bladder cancer patient-derived fibroblasts and MRC5, and
the bladder cancer cell layer includes any one or more of bladder cancer patient-derived bladder cancer cells, bladder cancer cell line T24, and bladder cancer cell line 5637.

3. The bladder cancer mimic of claim 1, wherein the bladder cancer mimic has a cylindrical shape.

4. The bladder cancer mimic of claim 1, wherein the endothelial cell layer, the fibroblast layer, and the bladder cancer cell layer are those obtained by 3D printing.

5. The bladder cancer mimic of claim 4, wherein the endothelial cell layer, the fibroblast layer, and the bladder cancer cell layer each have a filling rate of 5 to 25%.

6. The bladder cancer mimic of claim 4, wherein a permeate flow rate through the bladder cancer mimic is 10 to 30 μl/min.

7. A lab-on-a-chip comprising the bladder cancer mimic of claim 1.

8. A method for screening a substance for bladder cancer treatment, the method comprising:
treating the lab-on-a-chip of claim 7 with a candidate substance for bladder cancer treatment; and
comparing a group treated with the candidate substance with a group treated with a control substance.
